(19) United States
(12) Patent Application Publication
(10) Pub. No.: US 2026/0354750 A1
(43) Pub. Date: Nov. 12, 2026

(54) SPEECH FUNCTION ASSESSMENT

(71) Applicant: Hoffmann-La Roche Inc., Little Falls, NJ (US)

(72) Inventors: Thanneer Malai PERUMAL, Oberwil, Basel-Landschaft (CH); Raphael Marc ULLMANN, Basel (CH)

(21) Appl. No.: 19/118,347

(22) PCT Filed: Oct. 6, 2023
(86) PCT No.: PCT/EP2023/077744

§ 371 Date: Apr. 3, 2025

(30) Foreign Application Priority Data
Oct. 7, 2022 (EP) 22200420.2

(51) Int. Cl. A61B 5/00; G10L 25/03; G10L 25/66; G10L 25/84; G16H 50/20

(52) U.S. Cl. CPC A61B 5/4803; G10L 25/03; G10L 25/66; G10L 25/84; G16H 50/20

(57) ABSTRACT

A diagnostic device configured to assess the speech function of a subject, the diagnostic device comprising: at least one processor; a microphone; and a memory storing computer-readable instructions that, when executed by the at least one processor, cause the diagnostic device to: prompt the subject to perform a diagnostic task of speaking aloud; receive, via the microphone, audio data associated with the diagnostic task; extract, from the audio data, digital biomarker data associated with the speech function of the subject; and applying a speech function assessment model to the digital biomarker data, the speech function assessment model configured to generate an output indicative of the speech function of the subject.

### TECHNICAL FIELD OF THE INVENTION

The present invention relates to diagnostic devices and computer-implemented methods of assessing a subject's speech function.

### BACKGROUND TO THE INVENTION

Spinal muscular atrophy (SMA) is associated with bulbar weakness. People living with spinal muscular atrophy (SMA) report difficulty speaking loudly (e.g. to make themselves heard in a noisy environment), and may experience shortness of breath while speaking. It is important to note that speech impairments do not appear to be a meaningful aspect of health—in a Roche-sponsored qualitative study using structured interviews, 0% of SMA patients and their families mentioned speech among their top difficulties. However, among the 7 healthcare professionals (HCPs) surveyed, 3 ranked speech difficulties as being important. More generally, HCPs rated the need to measure bulbar abilities more than patients and their caregivers.

Moreover, the Scientific Advisory Working Group (SAWG) recommended that combining measurements from speech and respiration assessments could help detect worsening of bulbar function that might foreshadow critical events (such as aspirations). In other words, measurements derived from a speech-based assessment could serve as a leading indicator for hospitalizations.

Passage reading is often used for the assessment of motor speech disorders as a controlled and repeatable approximation of spontaneous, contextual speech¹. It has been shown that early signs of bulbar dysfunction in Amyotrophic Lateral Sclerosis (ALS) patients could be linked to a decrease in speech rate, more frequent speech pauses, and reduced articulatory precision^{2,3}.

In addition, since people with spinal muscular atrophy report difficulty speaking loudly, it is hypothesized that the sound pressure level⁴ of speech might be a further outcome measure. ¹ Patel, R., Connaghan, K., Franco, D., Edsall, E., Forgit, D., Olsen, L., Ramage, L., Tyler, E., & Russell, S. (2013). “The Caterpillar”: A Novel Reading Passage for Assessment of Motor Speech Disorders. American Journal of Speech-Language Pathology. https://doi.org/10.1044/1058-0360(2012/11-0134)² Allison, K. M., Yunusova, Y., Campbell, T. F., Wang, J., Berry, J. D., & Green, J. R. (2017). The diagnostic utility of patient-report and speech-language pathologists' ratings for detecting the early onset of bulbar symptoms due to ALS. Amyotrophic Lateral Sclerosis and Frontotemporal Degeneration, 18(5-6), 1-9. https://doi.org/10.1080/21678421.2017.1303515³ Stegmann, G. M., Hahn, S., Liss, J., Shefner, J., Rutkove, S., Shelton, K., Duncan, C. J., & Berisha, V. (2020). Early detection and tracking of bulbar changes in ALS via frequent and remote speech analysis NPJ Digital Medicine, 3(1), 132. https://doi.org/10.1038/s41746-020-00335-x⁴ This is often incorrectly referred to as “loudness”—loudness is a psychoacoustic term that refers to the subjective perception of sound pressure, and is affected by factors such the frequency-dependent sensitivity of human hearing, and masking effects that are used in audio compression schemes such as MP3. Unless these effects of human hearing are being modeled, the term level should be used.

In a remote patient monitoring setup using smartphones, the mouth-to-microphone distance is unknown, hence absolute speech level cannot be inferred from the recorded audio signal. However, assuming that the mouth-to-microphone distance remains constant during the task, we can measure changes in speech level over the task duration. This is based on the notion that during a prolonged reading effort, people with SMA might experience fatigue or shortness of breath that may manifest in a gradual decrease of speech level.

### SUMMARY OF THE INVENTION

The present invention provides a diagnostic device and computer-implemented methods of assessing speech function of a subject. The outputs may be useful in assessing bulbar function of a subject, and to track the status or progression of conditions affecting bulbar function, such as (but not exclusively) SMA.

More specifically, a first aspect of the invention provides a diagnostic device configured to assess the speech function of a subject, the diagnostic device comprising: at least one processor; a microphone; and a memory storing computer-readable instructions that, when executed by the at least one processor, cause the diagnostic device to: prompt the subject to perform a diagnostic task of speaking aloud; receive, via the microphone, audio data associated with the diagnostic task; extract, from the audio data, digital biomarker data associated with the speech function of the subject; and apply a speech function assessment model to the digital biomarker data, the speech function assessment model configured to generate an output indicative of the speech function of the subject based on the digital biomarker data.

By measuring speech function using a diagnostic device according to the first aspect of the present invention, it may be possible to track, effectively, the progress of various muscular disabilities such as SMA in a subject by active testing of the subject. In particular, the computer-readable instructions, when executed by the processor, may be further configured to cause the diagnostic device to map the output indicative of the speech function of the subject to a bulbar function assessment grade indicative of the bulbar function of the subject. As is described in detail later in this application, the diagnostic device according to the first aspect of the present invention may use the output indicative of the speech function and/or the bulbar function assessment grade to indicate and/or track the presence or progression of a muscular disability, such as SMA, in a subject or user.

In preferred implementations, the device is or comprises a smartphone. This is advantageous because smartphones are possessed by virtually everyone nowadays. By implementing a computer-implemented process such as the one described on a smartphone, a user need not attend e.g. a hospital or other clinical setting in order for the speech function to be measured. Other kinds of diagnostic device may be used, e. g. a tablet, a laptop computer, a desktop computer, or the like. Alternatively, the diagnostic device may be a dedicated speech function assessment device.

The computer-readable instructions, when executed by the processor, may cause the diagnostic device to prompt the subject to perform the diagnostic task by displaying text to be spoken aloud by the subject and/or audibly outputting a portion of the text to be spoken aloud by the subject. In some cases, in order better to acquaint the user with the text to be spoken aloud before they read it, the computer-readable instructions, when executed by the at least one processor, may further cause the diagnostic device to display the text for a preview period before receiving the audio data. During that time, the diagnostic device may be configured to indicate that audio data is not yet being received. This ensures that the subject does not begin reading until the dedicated time. In some cases, the diagnostic device may comprise a display component such as a touchscreen, which includes one or more sensors. Specifically, in response to a user tapping or touching the touchscreen at a location corresponding to a portion of the text to be spoken aloud, the diagnostic device may be configured to generate an audio output corresponding to the text in the area which the subject touched or tapped. This may aid the user to pronounce the words displayed at that part. This is particularly useful since declining pronunciation accuracy may be symptomatic of declining bulbar muscular function, discussed in more detail shortly. In response to a user input (either via one or more sensors in the touchscreen or otherwise) the subject may also be able to vary the font size at which the text to be spoken aloud is displayed. The computer-readable instructions, when executed by the processor, may further cause the diagnostic device to prompt the user speak aloud (e.g., to the read the displayed text aloud) with as few interruptions as possible. This may be particularly important, since increased numbers and/or durations of pauses in the subject's speech may be symptomatic of declining bulbar muscular function, discussed in more detail shortly.

We now discuss the nature of the digital biomarker data in more detail, and its extraction. Various types of digital biomarker data may be extracted from the recorded audio data, and the list of examples set out below is by no means exhaustive. Essentially, the types of digital biomarker parameterize various aspects of a subject's speech function, which may be affected by declining bulbar muscular function, e.g. as a result of SMA.

In some cases, the digital biomarker data may comprise a speech rate of the subject. This may be expressed in syllables, words, or phonemes per unit time (e.g. per minute or per second). In such cases, extracting the digital biomarker data may comprise applying a speech rate determination algorithm to the recorded audio data, the speech rate determination algorithm configured to determine a speech rate of the subject. The algorithm may be configured to detect words, syllables and/or phonemes within the recorded audio data, and to divide the number of detected words, syllables and/or phonemes by the total duration of active speech (i. e. excluding speech pauses) .

In some cases, the digital biomarker may comprise pronunciation accuracy, e.g. parameterized in the form of a distance between the acoustic features of the subject's speech in the recorded audio data and a reference (i.e. highly intelligible) speech recording. Appropriate pronunciation accuracy measures are set out in Janbakhshi et al. (2019)⁵, Bartelds et al. (2020)⁶, and Ullmann et al. (2015)⁷. In such cases, extracting the digital biomarker data may comprise applying a pronunciation accuracy determination algorithm to the recorded audio data, the pronunciation accuracy determination algorithm configured to determine a pronunciation accuracy of the subject. The algorithm may be an algorithm according to one of the previously-cited references. ⁵ Janbakhshi, P., Kodrasi, I., & Bourlard, H. (2019). Pathological Speech Intelligibility Assessment Based on the Short-time Objective Intelligibility Measure. ICASSP 2019-2019 IEEE International Conference on Acoustics, Speech and Signal Processing (ICASSP). https://doi.org/10.1109/icassp.2019.8683741⁶ Bartelds, M., Richter, C., Liberman, M., & Wieling, M. (2020). A New Acoustic-Based Pronunciation Distance Measure. Frontiers in Artificial Intelligence, 3, 39. https: //doi. org/10.3389/frai.2020.00039⁷ Ullmann, R., Magimai-Doss, M., & Bourlard, H. (2015). Objective Speech Intelligibility Assessment Through Comparison of Phoneme Class Conditional Probability Sequences. 2015 IEEE International Conference on Acoustics, Speech and Signal Processing (ICASSP), 4924-4928. https://doi.org/10.1109/icassp.2015.7178907

In some cases, the digital biomarker data may comprise information about pauses in the subject's speech in the recorded audio data. For example, the digital biomarker data may comprise the total duration of pauses in the recorded audio data, and/or a pausing rate (i.e. the total duration of pauses divided by the total duration of recorded audio data, or more specifically total duration of speech in the recorded audio data). In such cases, extracting the digital biomarker data may further comprise applying a pause identification algorithm to the recorded audio data, the pause identification algorithm configured to identify pauses within the recorded audio data. When the digital biomarker data comprises the total duration of pauses, extracting the digital biomarker may further comprise calculating the total duration of pauses in the recorded audio data based on the pauses identified by the pause identification algorithm. The pause identification algorithm may be configured to generate a plurality of timestamps indicating the beginning and end of each respective pause, and extracting the digital biomarker data may comprise calculating the total duration of pauses in the recorded audio data based on the generated plurality of timestamps.

When the digital biomarker data comprises the pausing rate, extracting the digital biomarker may further comprise calculating the total duration of pauses in the recorded audio data based on the pauses identified by the pause identification algorithm, and dividing it by the total duration of speech in the recorded audio data. The pause identification algorithm may be configured to generate a plurality of timestamps indicating the beginning and end of each respective pause, and extracting the digital biomarker data may comprise calculating the total duration of pauses in the recorded audio data based on the generated plurality of timestamps. Extracting the digital biomarker data may further comprise dividing the total duration of pauses by the total duration of speech in the recorded audio data.

In some cases, the digital biomarker data may comprise data indicative of a change in the level of the subject's speech over time. This may comprise a slope of a linear fit to the speech level over the duration of the task. Herein, the term “level” is used to refer to the absolute sound intensity of the speech. Such intensity may be measured either as a physical quantity such as sound pressure level (i.e., the local pressure deviation from the ambient atmospheric pressure), electrical voltage variation as measured by a microphone, or measurements of sound intensity that mimic the properties of human hearing such as A-weighted decibels (denoted “dB (A)”) or perceived loudness. It is desirable to assess, track, or measure the change in level because during a prolonged reading effort, people with SMA might experience fatigue or shortness of breath that may manifest in a gradual decrease of speech level. In implementations in which the level of speech is monitored, the mouth-to-microphone distance is preferably constant. Accordingly, the computer-readable instructions may, when executed by the processor, cause the diagnostic device to prompt the user to maintain a constant distance between their mouth and the microphone when carrying out the diagnostic task, or to place the diagnostic device in a predetermined location.

In the cases outlined in the previous paragraph, extracting digital biomarker data may comprise applying a speech level determination algorithm to the recorded audio data. In those cases, extracting the digital biomarker data may comprise determining a change in the level of the subject's speech over time based on the output of the speech level determination algorithm. In particular, extracting the digital biomarker data may comprise plotting speech level against time, generating a linear fit, and extracting, deriving, determining, or otherwise calculating a slope of the linear fit.

In some cases, the digital biomarker data may comprise one or more Mel-frequency cepstral coefficient (MFCC) values associated with the audio data. In particular, the digital biomarker data may comprise one or more low order MFCC values associated with the audio data, for example, the digital biomarker data may comprise one or more first Mel-frequency cepstral coefficient (MFCC 1) values associated with the audio data. Thus, extracting the digital biomarker data may comprise calculating one or more MFCC values, low order MFCC values or MFCC 1 values associated with the audio data.

When extracting digital biomarker data, it is desirable that the data is extracted from relevant portions of the recorded audio data. For example, there may be portions at the beginning, end, or intermediate portions of the recorded data which correspond to background noise, from before when the subject begins speaking, after the subject has finished speaking, or between two segments of the subject's speech. In other words, the recorded audio data may comprise a plurality of segments, and the diagnostic device may be configured to execute a preprocessing step comprising applying an active speech detection algorithm to the audio data, the active speech detection algorithm configured to classify the segments of the audio data into active speech segments and background noise segments. Classifying the segments of the audio data into active speech segments and background noise segments comprises generating timestamps indicating the beginning and end times of each respective active speech segment and background noise segment.

The extraction of digital biomarker data (as set out in the several preceding paragraphs) may then proceed only on the active speech segments.

In some examples, the computer-readable instructions, when executed by the processor, may cause the diagnostic device to execute a preprocessing step comprising re-sampling the received audio data to a predetermined sampling rate. This pre-processing step may be carried out before the pre-processing step of applying the active speech detection algorithm to the audio data, such that applying the active speech detection algorithm to the audio data may comprise applying the active speech detection algorithm to the re-sampled audio data. The predetermined sampling rate may be at least 10 kHz and no more than 20 kHz, for example, the predetermined sampling rate may be 16 kHz. In some cases, the active speech segments may comprise voiced sub-segments in which the vocal folds or vocal cords are actually vibrating, and un-voiced sub-segments, in which the vocal folds are not vibrating. For example, the subject's vocal folds may be vibrating while making the sound “a”, but not while making the sound “sh”. Typically, voiced sub-segments may be associated with vowel sounds. This is in contrast to background noise segments during which the subject is not speaking. Then, the diagnostic device may be configured to apply a voiced sub-segment detection algorithm to the active speech segments of the audio data, the voiced speech sub-segment detection algorithm configured to classify the sub-segments into voiced speech sub-segments and un-voiced speech sub-segments. Classifying the sub-segments of the active speech segments of the audio data into voiced speech segments and un-voiced speech segments comprises generating timestamps indicating the beginning and end times of each respective voiced speech sub-segment and un-voiced speech sub-segment. Extraction of digital biomarker data relating to e.g. speech level or pronunciation accuracy, or the one or more MFCC values, may then be executed only on the voiced speech sub-segments, for example to assess the phonatory function of the vocal folds, or the accuracy of vowel pronunciation, or the articulation range respectively.

When the digital biomarker data comprises one or more MFCC values, extracting the digital biomarker data may comprise extracting, from each voiced speech sub-segment, one or more MFCC values, such as one or more low order MFCC values, for example one or more MFCC 1 values. In this case, the speech function assessment model, applied to the digital biomarker data (i.e., the MFCC values), may be configured to calculate a variance of the MFCC values. The output indicative of the speech function of the subject may correspond to the variance of the MFCC values. For example, the output indicative of the speech function of the subject may correspond to the variance of low order MFCC values, such as the variance of MFCC 1 values.

Low-order MFCCs, such as MFCC 1 may describe the overall shape of a speech spectrum. During a voicing interval, the overall shape of the speech spectrum may be determined by the articulators and resonances within the vocal tract. Lower MFCC variance may therefore be indicative of reduced articulation range, as may be caused by bulbar weakness. As such, the variance of the low order MFCC values may be used to indicate the presence and/or progression of a muscular disability such as SMA, as discussed in further detail below.

In some cases the computer-readable instructions, when executed by the processor, may further cause the device to: receive, via the microphone, noise data; calculate, from the noise data, a background noise; and use the background noise to apply a correction to the audio data.

In some examples, the output indicative of the speech function of the subject may correspond to the digital biomarker data.

For example, the output indicative of the speech function may correspond to the speech rate, the pronunciation accuracy, the total duration of pauses, the pausing rate, or the change in the level of the subject's speech.

We now discuss how the output indicative of the speech function may be used to indicate a presence or a progression of a muscular disability, such as SMA. The computer-readable instructions, when executed by the at least one processor, may cause the diagnostic device to apply a clinical interpretation model to the output indicative of the speech function. The clinical interpretation model may be configured to output an indication of the presence or absence of a muscular disability, such as SMA, in the user, or an indication of the progression of a muscular disability in the user. The clinical interpretation model may be configured to compare the output indicative of the speech function to a predetermined value, and, based on the comparison, to output an indication of the presence or absence of the muscular disability, such as SMA.

In particular, the clinical interpretation model may be configured to determine whether the output indicative of the speech function is greater than a predetermined threshold. In some examples, the clinical interpretation model may be configured to, if it is determined that the output indicative of the speech function is greater than the predetermined threshold, to output an indication of the presence of a muscular disability (e.g., that the user is a PlwSMA), and/or if it is determined that the output indicative of the speech function is less than or equal to the predetermined threshold, to output an indication of the absence of the muscular disability. In other examples, the clinical interpretation model may be configured to, if it is determined that the output indicative of the speech function is less than the predetermined threshold, to output an indication of the presence of a muscular disability (e.g., that the user is a PlwSMA), and/or if it is determined that the output indicative of the speech function is greater than or equal to the predetermined threshold, to output an indication of the absence of the muscular disability. This may be the case when the output indicative of the speech function is the variance of low order MFCC values. When the output indicative of the speech function is the variance of low order MFCC values, the predetermined threshold may be 10,000 or less. The predetermined threshold may be 9,000 or less, or 8,000 or less. The predetermined threshold may be 6,000 or more. The predetermined threshold may be 7,000 or more, or 8,000 or more.

For example, PlwSMA may demonstrate a significantly lower MFCC 1 variance as compared to healthy subjects due to bulbar weakness of PlwSMA, as discussed above.

A second aspect of the invention provides a computer-implemented method of assessing the speech function of a subject, the computer-implemented method comprising the steps of: prompting the subject to perform a diagnostic task of speaking aloud; receiving, via a microphone, audio data associated with the diagnostic task; extracting, from the audio data, digital biomarker data associated with the speech function of the subject; and applying a speech function assessment model to the digital biomarker data, the speech function assessment model configured to generate an output indicative of the speech function of the subject based on the digital biomarker data. In preferred cases, the computer-implemented method of the second aspect of the invention is executed by a processor of a diagnostic device such as the diagnostic device of the first aspect of the invention. It will be appreciated that the optional features set out above, in respect of the first aspect of the invention, apply equally well to the second aspect of the invention except where context clearly dictates otherwise, or whether such a combination of features is clearly technically incompatible.

A third aspect of the invention provides a computer program comprising instructions which when executed by a processor of a computer (or other suitable data processing device) cause the processor to execute the computer-implemented method of the second aspect of the invention. A further aspect of the invention provides a computer-readable storage medium having stored thereon the computer program of the third aspect of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 is a diagram of an example environment in which a diagnostic device for assessing speech function of a subject is provided.

FIG. 2 is a flow diagram of a computer-implemented method for assessing the speech function of a user.

FIG. 3 is a flow diagram of a computer-implemented method for determining an indication of the presence or absence of a muscular disability, such as SMA.

FIG. 4 is a plot showing MFCC 1 variances calculated for PlwSMA and for healthy individuals.

FIG. 5 illustrates one example of a network architecture and data processing device that may be used to implement one or more illustrative aspects described herein.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

In the following description of various aspects, reference is made to the accompanying drawings, which form a part hereof, and in which is shown by way of illustration various embodiments in which aspects described herein may be practiced. It is to be understood that other aspects and/or embodiments may be utilized, and structural and functional modifications may be made without departing from the scope of the described aspects and embodiments.

Aspects described herein are capable of other embodiments and of being practiced or being carried out in various ways. Also, it is to be understood that the phraseology and terminology used herein are for the purpose of description and should not be regarded as limiting. Rather, the phrases and terms used herein are to be given their broadest interpretation and meaning. The use of “including” and “comprising” and variations thereof is meant to encompass the items listed Thereafter and equivalents thereof as well as additional items and equivalents thereof. The use of the terms “mounted,” “connected,” “coupled,” “positioned,” “engaged” and similar terms, is meant to include both direct and indirect mounting, connecting, coupling, positioning and engaging.

Systems, methods and devices described herein provide a diagnostic device and computer-implemented methods for assessing, measuring, or determining the speech function of a patient, for example a patient suffering from a muscular disability, such as in particular SMA. In some cases, the diagnostic device may be in the form of a mobile device, in particular a smartphone, on which a particular software application is installed. The software application may be configured to execute (or cause the processor of the mobile device) the corresponding computer-implemented method.

In some cases, the diagnostic obtains or receives sensor data from one or more sensors associated with the mobile device as the subject interacts with the software application using the mobile device. In some cases, the sensors may be within the mobile device. In some cases, the data indicative of speech function of a subject is derived, calculated, or extracted from the received or obtained sensor data. In some cases, the assessment of the symptom severity and progression of a muscular disability, in particular SMA, in the subject may be determined based on the extracted sensor features.

In implementations of the present invention, the diagnostic device may prompt the subject to perform diagnostic tasks. In some cases, the diagnostic tasks are anchored in or modelled after established methods and standardized tests. In some cases, in response to the subject performing the diagnostic task, the diagnostic obtains or receives sensor data via one or more sensors. In some cases, the sensors may be within a mobile device or wearable sensors worn by the subject. In some cases, sensor features associated with the symptoms of a muscular disability, in particular SMA, are extracted from the received or obtained sensor data. In some cases, the assessment of the symptom severity and progression of a muscular disability, in particular SMA, in the subject is determined based on the extracted features of the sensor data. Assessments of symptom severity and progression of a muscular disability, in particular SMA, using diagnostics according to the present disclosure correlate sufficiently with the assessments based on clinical results and may thus replace clinical subject monitoring and testing. Example diagnostics according to the present disclosure may be used in an out of clinic environment, and therefore have advantages in cost, ease of subject monitoring and convenience to the subject.

This facilitates frequent, in particular daily, subject monitoring and testing, resulting in a better understanding of the disease stage and provides insights about the disease that are useful to both the clinical and research community. An example diagnostic according to the present disclosure can provide earlier detection of even small changes in speech function of a subject which can be indicative of the presence or progression of muscular disabilities, in particular SMA, in a subject and can therefore be used for better disease management including individualized therapy.

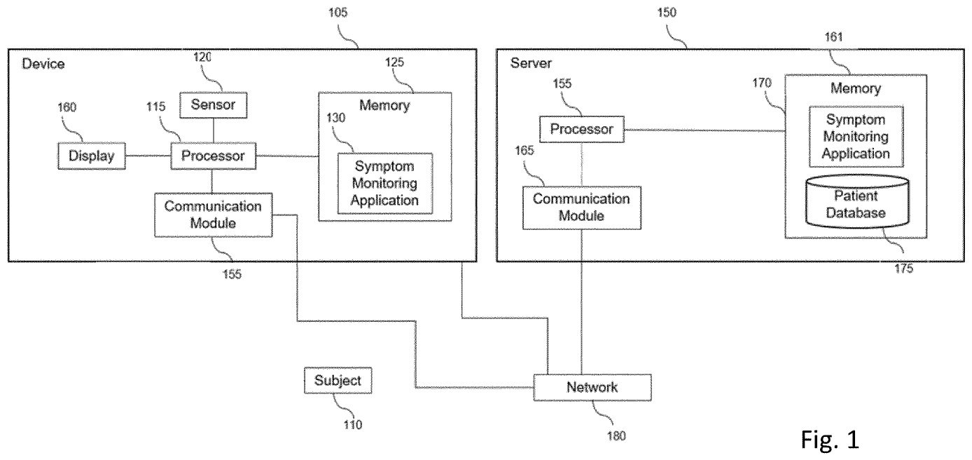

FIG. 1 is a diagram of an example environment in which a diagnostic device 105 for assessing speech function of a subject 110. In some cases, the device 105 may be a smartphone, a smartwatch or other mobile computing device. The device 105 includes a display screen 160. In some cases, the display screen 160 may be a touchscreen. The device 105 includes at least one processor 115 and a memory 125 storing computer-instructions for a symptom monitoring application 130 that, when executed by the at least one processor 115, cause the device 105 to assess speech function of a subject. The device 105 receives a plurality of sensor data via one or more sensors associated with the device 105. In some cases, the one or more sensors associated with the device is at least one of a sensor disposed within the device or a sensor worn by the subject and configured to communicate with the device. In FIG. 1, the sensors associated with the device 105 include a first sensor 120 such as a microphone which is located in device 105.

The device 105 extracts, from the received first sensor data, digital biomarker data, which can be used to determine speech function of a subject.

The device 105 determines the speech function of a subject 110 based on the extracted features. In some cases, the device 105 sends the extracted features over a network 180 to a server 150. In some cases, the device 105 sends the first sensor data over the network 180 to the server 150. The server 150 includes at least one processor 155 and a memory 161 storing computer-instructions for a symptom assessment application 170 that, when executed by the server processor 155, cause the processor 155 to determine speech function of a subject 110 based on the extracted features received by the server 150 from the device 105. In some cases, the symptom assessment application 170 may cause the processor 115 to extract the features from the sensor data received from the device 105. In some cases, the symptom assessment application 170 may determine the speech function of a subject 110 based on the extracted features of the sensor data, which may be received from the device 105, and a subject database 175 stored in the memory 160. In some cases, the subject database 175 may include subject and/or clinical data. In some cases, the subject database 175 may include in-clinic and sensor-based measures of the speech function. In some cases, the subject database 175 may be independent of the server 150. In some cases, the server 150 sends the determined speech function of a subject 110 to the device 105. In some cases, the device 105 may output the speech function of a subject 110. In some cases, the device 105 may communicate information to the subject 110 based on the assessment. In some cases, the assessment of speech function of a subject 110, may be communicated to a clinician that may determine individualized therapy for the subject 110 based on the assessment.

In some cases, the computer-instructions for the symptom monitoring application 130, when executed by the at least one processor 115, cause the device 105 to determine the speech function of a subject 110 based on active testing of the subject 110. The device 105 prompts the subject 110 to perform one or more tasks. In some cases, prompting the subject to perform the one or more diagnostic tasks includes prompting the subject to make a continuous “aaah” sound for as long as possible.

In response to the subject 110 performing the one or more diagnostic tasks, the diagnostic device 105 receives a plurality of sensor data via the one or more sensors associated with the device 105. The device 105 extracts, from the received sensor data various digital biomarker data, from which an assessment of speech function of a subject 110 may be made. The symptoms of a muscular disability, in particular SMA in the subject 110 may include a symptom affecting the speech function of a subject 110.

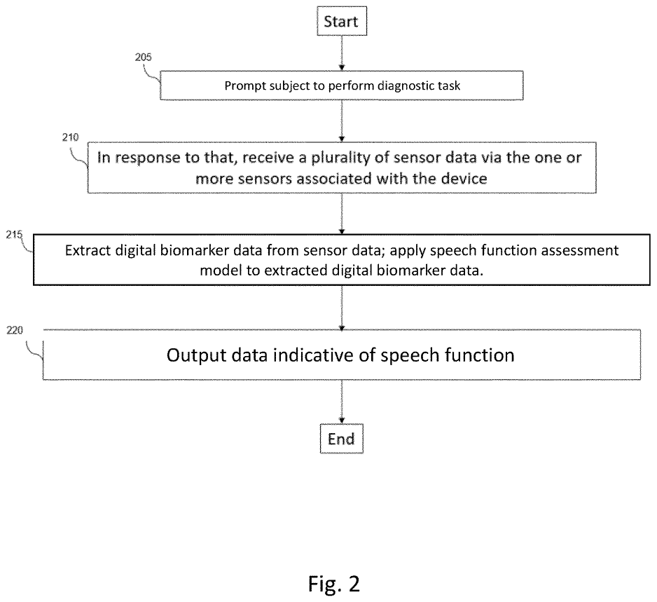

FIG. 2 illustrates an example method for assessing the speech function of a subject 110 based on active testing of the subject using the example device 105 of FIG. 1. While FIG. 2 is described with reference to FIG. 1, it should be noted that the method steps of FIG. 2 may be executed by other systems.

The computer-implemented method includes, in step 205, prompting the subject to perform a diagnostic task as outlined above. The method includes receiving, in response to the subject performing the one or more tasks, a plurality of sensor data, via e.g. a microphone (step 210).

Then, in step 215, digital biomarker data is extracted from the sensor data, and a speech function assessment model is applied to the digital biomarker data.

In step 220, data indicative of a speech function of a subject is output, e.g. by the processor 107 generating instructions, which when executed by the display component 160 of the device 105 cause the display component 160 to display an output indicative of the speech function of a subject.

Alternatively, the calculated data indicative of the speech function of a subject may be transmitted to a server 150, as outlined elsewhere in this application.

As discussed above, assessments of symptom severity and progression of a muscular disability, in particular SMA, using diagnostics according to the present disclosure, correlate sufficiently with the assessments based on clinical results and may thus replace clinical subject monitoring and testing.

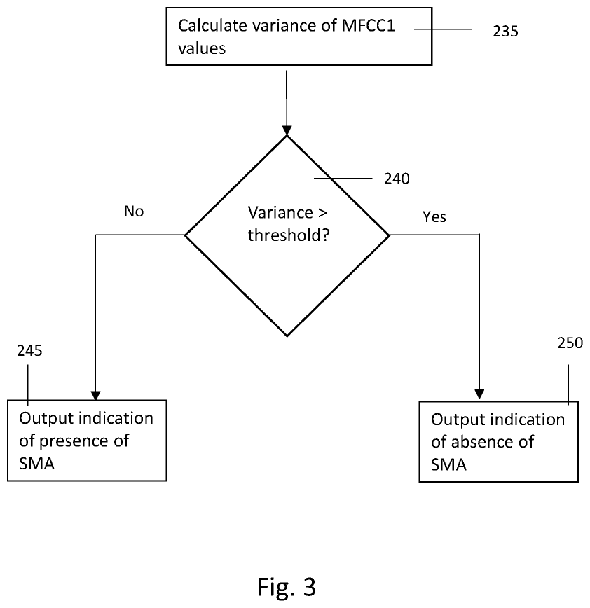

FIG. 3 illustrates an example method for determining an indication of the presence or absence of SMA in a subject based on active testing of the subject using the example device 105 of FIG. 1. While FIG. 3 is described with reference to FIG. 1, it should be noted that the method steps of FIG. 3 may be executed by other systems. The computer-implemented method includes, in step 235, calculating a variance of MFCC 1 values of the audio data. This variance corresponds to the output indicative of the speech function in the method described with reference to FIG. 2. In step 240, the computer-implemented method includes determining whether the calculated variance is greater than a predetermined threshold. If the calculated variance is determined to be greater than the predetermined threshold, in step 250 the computer-implemented method includes outputting an indication of the absence of SMA. If the calculated variance is determined to less than or equal to the predetermined threshold, in step 245 the computer-implemented method includes outputting an indication of the presence of SMA.

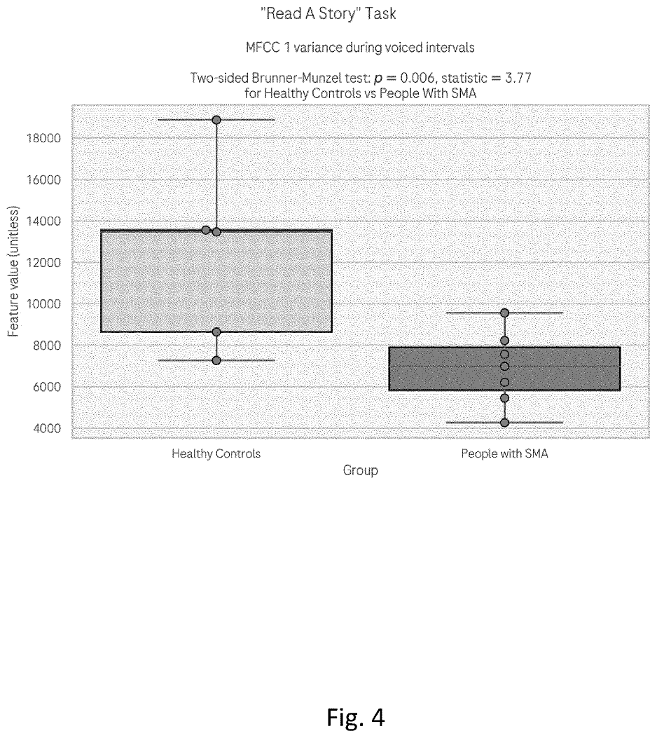

For example, the predetermined threshold may be approximately 9,000. That is, a variance of >9,000 may indicate that a user is healthy individual, and a variance of ≤9,000 may indicate that a user is a PlwSMA. This may be explained with reference to FIG. 4. FIG. 4 is a plot showing calculated variances of MFCC 1 values for PlwSMA and for healthy individuals. This plot shows that a majority of PlwSMA may have test results of a variance of MFCC 1 values of ≤9,000, whereas a majority of healthy individuals may have test results of a variance of MFCC 1 values ofs>9,000.

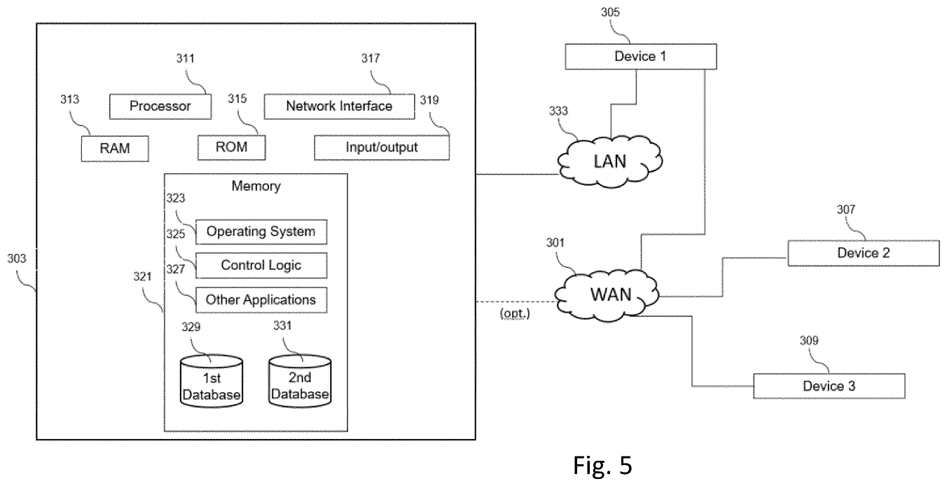

FIG. 5 illustrates an example of a network architecture and data processing device that may be used to implement one or more illustrative aspects described herein, such as the aspects described in FIGS. 1 and 2. Various network nodes 303, 305, 307, and 309 may be interconnected via a wide area network (WAN) 301, such as the Internet. Other networks may also or alternatively be used, including private intranets, corporate networks, LANs, wireless networks, personal networks (PAN), and the like. Network 301 is for illustration purposes and may be replaced with fewer or additional computer networks. A local area network (LAN) may have one or more of any known LAN topology and may use one or more of a variety of different protocols, such as Ethernet. Devices 303, 305, 307, 309 and other devices (not shown) may be connected to one or more of the networks via twisted pair wires, coaxial cable, fibre optics, radio waves or other communication media.

The term “network” as used herein and depicted in the drawings refers not only to systems in which remote storage devices are coupled together via one or more communication paths, but also to stand-alone devices that may be coupled, from time to time, to such systems that have storage capability. Consequently, the term “network” includes not only a “physical network” but also a “content network,” which is comprised of the data-attributable to a single entity which resides across all physical networks.

The components may include data server 303, web server 305, and client computers 307, 309. Data server 303 provides overall access, control and administration of databases and control software for performing one or more illustrative aspects described herein. Data server 303 may be connected to web server 305 through which users interact with and obtain data as requested. Alternatively, data server 303 may act as a web server itself and be directly connected to the Internet. Data server 303 may be connected to web server 305 through the network 301 (e.g., the Internet), via direct or indirect connection, or via some other network. Users may interact with the data server 303 using remote computers 307, 309, e.g., using a web browser to connect to the data server 303 via one or more externally exposed web sites hosted by web server 305. Client computers 307, 309 may be used in concert with data server 303 to access data stored therein, or may be used for other purposes. For example, from client device 307 a user may access web server 305 using an Internet browser, as is known in the art, or by executing a software application that communicates with web server 305 and/or data server 303 over a computer network (such as the Internet). In some cases, the client computer 307 may be a smartphone, smartwatch or other mobile computing device, and may implement a diagnostic device, such as the device 105 shown in FIG. 1. In some cases, the data server 303 may implement a server, such as the server 150 shown in FIG. 1.

Servers and applications may be combined on the same physical machines, and retain separate virtual or logical addresses, or may reside on separate physical machines. FIG. 1 illustrates just one example of a network architecture that may be used, and those of skill in the art will appreciate that the specific network architecture and data processing devices used may vary, and are secondary to the functionality that they provide, as further described herein. For example, services provided by web server 305 and data server 303 may be combined on a single server.

Each component 303, 305, 307, 309 may be any type of known computer, server, or data processing device. Data server 303, e.g., may include a processor 311 controlling overall operation of the rate server 303. Data server 303 may further include RAM 313, ROM 315, network interface 317, input/output interfaces 319 (e.g., keyboard, mouse, display, printer, etc.) , and memory 321. I/O 319 may include a variety of interface units and drives for reading, writing, displaying, and/or printing data or files. Memory 321 may further store operating system software 323 for controlling overall operation of the data processing device 303, control logic 325 for instructing data server 303 to perform aspects described herein, and other application software 327 providing secondary, support, and/or other functionality which may or may not be used in conjunction with other aspects described herein. The control logic may also be referred to herein as the data server software 325. Functionality of the data server software may refer to operations or decisions made automatically based on rules coded into the control logic, made manually by a user providing input into the system, and/or a combination of automatic processing based on user input (e.g., queries, data updates, etc.).

Memory 321 may also store data used in performance of one or more aspects described herein, including a first database 329 and a second database 331. In some cases, the first database may include the second database (e.g., as a separate table, report, etc.). That is, the information can be stored in a single database, or separated into different logical, virtual, or physical databases, depending on system design. Devices 305, 307, 309 may have similar or different architecture as described with respect to device 303. Those of skill in the art will appreciate that the functionality of data processing device 303 (or device 305, 307, 309) as described herein may be spread across multiple data processing devices, for example, to distribute processing load across multiple computers, to segregate transactions based on geographic location, user access level, quality of service (QoS), etc.

One or more aspects described herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer-readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects, and such data structures are contemplated within the scope of computer executable instructions and computer-usable data described herein.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A diagnostic device configured to assess the speech function of a subject, the diagnostic device comprising:
at least one processor;
a microphone; and
a memory storing computer-readable instructions that, when executed by the at least one processor, cause the diagnostic device to:
prompt the subject to perform a diagnostic task of speaking aloud;
receive, via the microphone, audio data associated with the diagnostic task;
extract, from the audio data, digital biomarker data associated with the speech function of the subject; and
applying a speech function assessment model to the digital biomarker data, the speech function assessment model configured to generate an output indicative of the speech function of the subject.

2. A diagnostic device according to claim 1, wherein:
the computer-readable instructions, when executed by the processor, cause the diagnostic device to prompt the subject to perform the diagnostic task by displaying text to be spoken aloud by the subject or audibly outputting a portion of the text to be spoken aloud by the subject.

3. A diagnostic device according to claim 1, wherein:
the digital biomarker data comprises a speech rate of the subject in units of syllables, words, or phonemes per unit time; and
extracting the digital biomarker data comprises applying a speech rate determination algorithm to the recorded audio data, the speech rate determination algorithm configured to determine a speech rate of the subject.

4. A diagnostic device according to claim 1, wherein:
the digital biomarker data comprises pronunciation accuracy; and
extracting the digital biomarker data comprises applying a pronunciation accuracy determination algorithm to the recorded audio data, the pronunciation accuracy determination algorithm configured to determine a pronunciation accuracy of the subject.

5. A diagnostic device according to claim 1, wherein:
extracting the digital biomarker data comprises applying a pause identification algorithm to the recorded audio data, the pause identification algorithm configured to identify pauses within the recorded audio data.

6. A diagnostic device according to claim 5, wherein:
the digital biomarker data comprises a total duration of pauses in the recorded audio data; and
extracting the digital biomarker data further comprises calculating the total duration of pauses in the recorded audio data based on the pauses identified by the pause identification algorithm.

7. A diagnostic device according to claim 1, wherein:
extracting the digital biomarker data comprises applying a speech level determination algorithm to the recorded audio data.

8. A diagnostic device according to claim 7, wherein:
the digital biomarker data comprises data indicative of a change in the level of the subject's speech over time; and
extracting the digital biomarker data comprises determining a change in the level of the subject's speech over time based on the output of the speech level determination algorithm.

9. A diagnostic device according to claim 1, wherein the computer-readable instructions, when executed by the processor, cause the diagnostic device to execute a preprocessing step, the preprocessing step comprising applying an active speech detection algorithm to the audio data, the active speech detection algorithm configured to classify segments of the audio data into active speech segments and background noise segments.

10. A diagnostic device according to claim 9, wherein the computer-readable instructions, when executed by the processor, cause the diagnostic device to apply a voiced sub-segment detection algorithm to the active speech segments of the audio data, the voiced speech sub-segment detection algorithm configured to classify the sub-segments into voiced speech sub-segments and un-voiced speech sub-segments.

11. A diagnostic device according to claim 10, wherein extracting the digital biomarker data comprises extracting, from each voiced speech sub-segment, one or more low order Mel-frequency cepstral coefficient (MFCC) values.

12. A diagnostic device according to claim 11, wherein the speech function assessment model is configured to calculate a variance of the low order MFCC values such that the output indicative of the speech function of the subject corresponds to the variance of the low order MFCC values.

13. A diagnostic device according to claim 1 wherein the computer-readable instructions, when executed by the at least one processor, cause the diagnostic device to apply a clinical interpretation model to the output indicative of the speech function, wherein the clinical interpretation model outputs an indication of the presence or absence of a muscular disability.

14. A diagnostic device according to claim 13, wherein the clinical interpretation model is configured to compare the output indicative of the speech function to a predetermined value, and, based on the comparison, to output an indication of the presence or absence of the muscular disability.

15. A diagnostic device according to claim 14, wherein the clinical interpretation model is configured to:
determine whether the output indicative of the speech function is greater than a predetermined threshold; and,
if it is determined that the output indicative of the speech function is greater than the predetermined threshold, to output an indication of the presence of a muscular disability; and,
if it is determined that the output indicative of the speech function is less than or equal to the predetermined threshold, to output an indication of the absence of the muscular disability.

16. A diagnostic device according to claim 14, wherein the clinical interpretation model is configured to:
determine whether the output indicative of the speech function is less than a predetermined threshold; and,
if it is determined that the output indicative of the speech function is less than the predetermined threshold, to output an indication of the presence of a muscular disability; and,
if it is determined that the output indicative of the speech function is greater than or equal to the predetermined threshold, to output an indication of the absence of the muscular disability.

17. A diagnostic device according to claim 16, wherein the predetermined threshold is 10,000 or less, and 6,000 or more.

18. A computer-implemented method of assessing the speech function of a subject, the computer-implemented method comprising the steps of:
prompting the subject to perform a diagnostic task of speaking aloud;
receiving, via a microphone, audio data associated with the diagnostic task;
extracting, from the audio data, digital biomarker data associated with the speech function of the subject; and
applying a speech function assessment model to the digital biomarker data, the speech function assessment model configured to generate an output indicative of the speech function of the subject based on the digital biomarker data.

19. A computer-implemented method according to claim 18, wherein the computer-implemented method further comprises the steps of:
applying a clinical interpretation model to the output indicative of the speech function, wherein the clinical interpretation model outputs an indication of the presence or absence of a muscular disability, or an indication of the progression of a muscular disability.

20. (canceled)

21. A computer-implemented method according to claim 18, wherein the steps of prompting the subject and receiving the audio data are carried out by a processor of a diagnostic device, and wherein the steps of extracting the digital biomarker data and applying the speech function assessment model are carried out by a processor of a server, wherein the diagnostic device is configured to transmit the audio data to the server, and wherein the diagnostic device comprises:
at least one processor;
a microphone; and
a memory storing computer-readable instructions that, when executed by the at least one processor, cause the diagnostic device to:
prompt the subject to perform a diagnostic task of speaking aloud;
receive, via the microphone, audio data associated with the diagnostic task.
